(19) United States
(12) Patent Application Publication
(10) Pub. No.: US 2017/0265778 A1
(43) Pub. Date: Sep. 21, 2017

(54) Barometric Pressure Sensor Spirometer

(71) Applicant: MySpirometer LLC, Mount Pleasant, SC (US)

(72) Inventors: Ryan Reichlyn, Carlsbad, CA (US); Ryan Roberts, Carlsbad, CA (US)

(21) Appl. No.: 15/366,750

(22) Filed: Dec. 1, 2016

Related U.S. Application Data
(60) Provisional application No. 62/310,096, filed on Mar. 18, 2016.

(51) Int. Cl. A61B 5/091; A61B 5/097

(52) U.S. Cl. CPC A61B 5/091; A61B 5/097; A61B 2560/0214; A61B 2560/0443

(57) ABSTRACT

A spirometer that includes a sensor and a specifically designed flow chamber to create a pressure change and produce spirometry test results is disclosed. In one embodiment, the sensor comprises an absolute barometric pressure sensor. The spirometer of the present disclosure includes a single sensor positioned between the inlet of a flow chamber and a barrier to compute spirometry values with barometric pressure change. Pressure, humidity, and temperature are features that are built into the sensor. These sensor features and values assist in final computational spirometry test values as users complete test maneuvers.

### CROSS-REFERENCE TO RELATED APPLICATION

The present application claims priority to U.S. Provisional Application No. 62/310,096, filed Mar. 18, 2016, entitled “Barometric Pressure Sensor Spirometer”, the entire disclosure of which is hereby incorporated by reference.

### BACKGROUND OF THE INVENTION

1. Field of the Disclosure

The present disclosure relates generally to devices for monitoring lung function. More particularly, the present disclosure relates to devices for monitoring lung function including a barometric pressure sensor as a primary means of measuring pulmonary function.

2. Description of the Related Art

A spirometer is a device that monitors respiration. For example, a spirometer measures the volume of air inspired and expired by the lungs. A spirometer can be used for the diagnosis and monitoring of pulmonary function. A spirometer may be used for the diagnosis of a person's respiratory condition and to monitor the condition of the lungs on a regular basis.

### SUMMARY OF THE INVENTION

The present disclosure provides a spirometer that includes a sensor and a specifically designed flow chamber to create a pressure change and produce spirometry test results. In one embodiment, the sensor comprises an absolute barometric pressure sensor. The spirometer of the present disclosure includes a single sensor positioned between the inlet of a flow chamber and a barrier to compute spirometry values with barometric pressure change. Pressure, humidity, and temperature are features that are built into the sensor. These sensor features and values assist in final computed spirometry test values as users complete test maneuvers.

In accordance with an embodiment of the present invention, a spirometer includes a flow chamber defining a pathway, the flow chamber having an inlet and an outlet; a barrier defining a volumetric area, the barrier positioned within the flow chamber to obstruct a portion of the pathway; and a sensor positioned between the inlet and the barrier.

In one configuration, the sensor comprises an absolute barometric pressure sensor. In another configuration, the flow chamber includes a lip extending within the pathway adjacent the inlet. In yet another configuration, the spirometer includes a mouthpiece having a first portion and a second portion, the second portion having an end wall, the second portion of the mouthpiece received within the inlet of the flow chamber such that the end wall contacts the lip of the flow chamber. In one configuration, with the mouthpiece received within the inlet of the flow chamber, there are no gaps between the mouthpiece and the flow chamber. In another configuration, the spirometer includes a disposable mouthpiece sheath that protectively covers the mouthpiece. In yet another configuration, the mouthpiece includes a tapered portion. In one configuration, the spirometer includes a printed circuit board in communication with the sensor. In another configuration, the spirometer includes a pressure seal between the flow chamber and the printed circuit board. In yet another configuration, the spirometer includes a button in communication with the printed circuit board, the button transitionable between an on position and an off position. In one configuration, the spirometer includes a battery. In another configuration, the spirometer includes a housing that protectively covers the flow chamber, the sensor, the printed circuit board, and the battery. In yet another configuration, the housing comprises a top housing, a bottom housing, a first side housing, and a second side housing. In one configuration, the spirometer includes a cap that protectively covers the mouthpiece, the cap removably connectable to a portion of the housing.

In accordance with another embodiment of the present invention, a spirometer includes a flow chamber defining a pathway, the flow chamber having an inlet, an outlet, and a lip extending within the pathway adjacent the inlet; a barrier defining a volumetric area, the barrier positioned within the flow chamber to obstruct a portion of the pathway; a sensor positioned between the inlet and the barrier, wherein the sensor comprises an absolute barometric pressure sensor; and a mouthpiece having a first portion and a second portion, the second portion having an end wall, the second portion of the mouthpiece received within the inlet of the flow chamber such that the end wall contacts the lip of the flow chamber.

In one configuration, with the mouthpiece received within the inlet of the flow chamber, there are no gaps between the mouthpiece and the flow chamber. In another configuration, the spirometer includes a disposable mouthpiece sheath that protectively covers the mouthpiece. In yet another configuration, the mouthpiece includes a tapered portion. In one configuration, the spirometer includes a printed circuit board in communication with the sensor. In another configuration, the spirometer includes a pressure seal between the flow chamber and the printed circuit board. In yet another configuration, the spirometer includes a button in communication with the printed circuit board, the button transitionable between an on position and an off position. In one configuration, the spirometer includes a battery. In another configuration, the spirometer includes a housing that protectively covers the flow chamber, the sensor, the printed circuit board, and the battery. In yet another configuration, the housing comprises a top housing, a bottom housing, a first side housing, and a second side housing. In one configuration, the spirometer includes a cap that protectively covers the mouthpiece, the cap removably connectable to a portion of the housing.

In accordance with another embodiment of the present invention, a spirometer includes a flow chamber defining a pathway, the flow chamber having an inlet and an outlet; a barrier defining a volumetric area, the barrier positioned within the flow chamber to obstruct a portion of the pathway; a sensor positioned between the inlet and the barrier; and a membrane that protectively covers the sensor, the membrane movable relative to a portion of the flow chamber.

In one configuration, the sensor comprises an absolute barometric pressure sensor.

In accordance with another embodiment of the present invention, a combination includes a spirometer comprising: a flow chamber defining a pathway, the flow chamber having an inlet and an outlet; a barrier defining a volumetric area, the barrier positioned within the flow chamber to obstruct a portion of the pathway; and a sensor positioned between the inlet and the barrier; and a mobile device in communication with the spirometer, the mobile device having a secondary sensor.

In one configuration, the sensor comprises an absolute barometric pressure sensor.

In accordance with another embodiment of the present invention, a spirometer includes a flow chamber defining a pathway, the flow chamber having an inlet and an outlet; a barrier defining a volumetric area, the barrier positioned within the flow chamber to obstruct a portion of the pathway; a sensor positioned between the inlet and the barrier; and an accelerometer that detects movement.

In one configuration, the sensor comprises an absolute barometric pressure sensor.

In accordance with another embodiment of the present invention, a spirometer includes a flow chamber defining a pathway, the flow chamber having an inlet and an outlet; a barrier defining a volumetric area, the barrier positioned within the flow chamber to obstruct a portion of the pathway; a sensor positioned between the inlet and the barrier; and a flap movably connected to a portion of the barrier, the flap movable between a closed position and an open position.

In one configuration, the sensor comprises an absolute barometric pressure sensor. In another configuration, with the flap in the closed position, the flap covers a portion of the pathway adjacent the barrier. In yet another configuration, with the flap in the open position, the flap is pivoted away from the barrier. In one configuration, the flap is formed of a flexible material. In another configuration, the barrier and the flap form a unitary component, the flap movably connected to the barrier via a living hinge.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a first exploded, perspective view of a spirometer in accordance with an embodiment of the present invention.

FIG. 2 is a second exploded, perspective view of a spirometer in accordance with an embodiment of the present invention.

FIG. 3 is a third exploded, perspective view of a spirometer in accordance with an embodiment of the present invention.

FIG. 4 is a fourth exploded, perspective view of a spirometer in accordance with an embodiment of the present invention.

FIG. 5 is a first cross-sectional view of a spirometer in accordance with an embodiment of the present invention.

FIG. 6 is a second cross-sectional view of a spirometer in accordance with an embodiment of the present invention.

FIG. 7 is a perspective view of a flow chamber of a spirometer in accordance with an embodiment of the present invention.

FIG. 8 is a cross-sectional view of a flow chamber of a spirometer in accordance with an embodiment of the present invention.

FIG. 9 is a perspective view of a protective cap of a spirometer in accordance with an embodiment of the present invention.

FIG. 10 is a perspective view of a mouthpiece of a spirometer in accordance with an embodiment of the present invention.

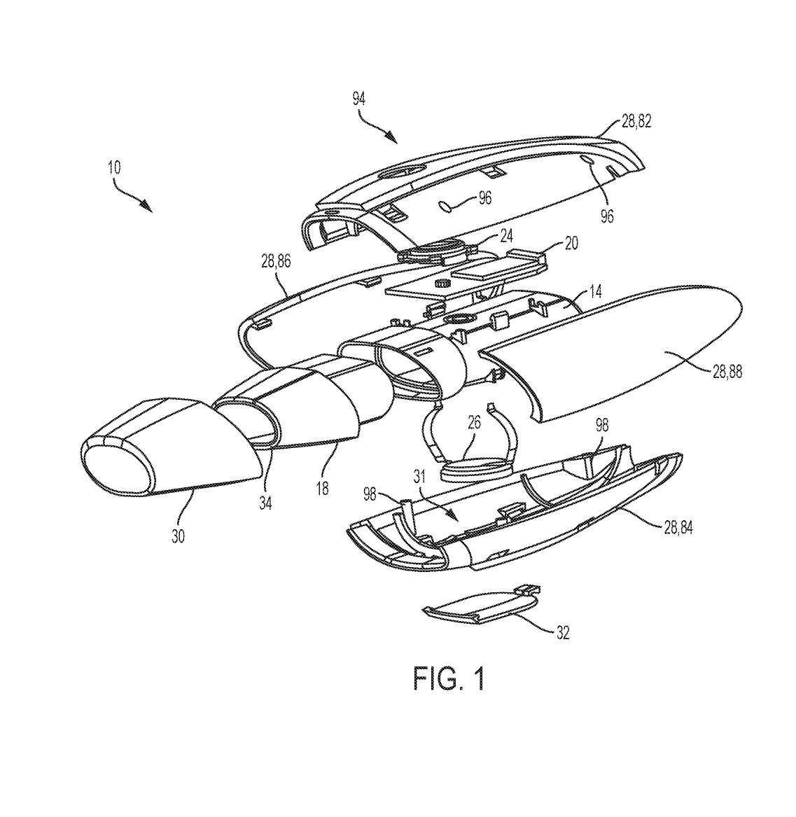

FIG. 1 is an elevation view of a printed circuit board of a spirometer in accordance with an embodiment of the present invention.

FIG. 12 is a perspective view of a mouthpiece of a spirometer in accordance with another embodiment of the present invention.

FIG. 13 is a perspective view of a spirometer in accordance with an embodiment of the present invention.

FIG. 14 is a perspective view of a spirometer with a protective cap removed from a mouthpiece in accordance with an embodiment of the present invention.

FIG. 15 is a perspective view of a spirometer in accordance with an embodiment of the present invention.

FIG. 16 is an elevation view of a spirometer in accordance with an embodiment of the present invention.

FIG. 17 is an elevation view of a spirometer with a protective cap covering a mouthpiece in accordance with an embodiment of the present invention.

FIG. 18 is a perspective view of a spirometer being held by a user in accordance with an embodiment of the present invention.

FIG. 19 is a side elevation view of a spirometer in accordance with an embodiment of the present invention.

FIG. 20 is an elevation view of a spirometer in accordance with an embodiment of the present invention.

FIG. 21 is an elevation view of a spirometer with a protective cap removed from a mouthpiece in accordance with an embodiment of the present invention.

FIG. 22 is an elevation view of a spirometer in accordance with another embodiment of the present invention.

FIG. 23 is an elevation view of a spirometer with a protective cap removed from a mouthpiece in accordance with another embodiment of the present invention.

FIG. 24 is an elevation view of a spirometer with a membrane protectively covering a sensor in accordance with an embodiment of the present invention.

FIG. 25 is a schematic representation of a spirometer in communication with a mobile device in accordance with an embodiment of the present invention.

FIG. 26 is a perspective view of a spirometer having an accelerometer in accordance with an embodiment of the present invention.

FIG. 27 is an exploded, perspective view of a barrier and a flap of a spirometer in accordance with an embodiment of the present invention.

FIG. 28 is an assembled, perspective view of a barrier and a flap of a spirometer in accordance with an embodiment of the present invention.

FIG. 29 is a cross-sectional view of a spirometer with a flap in a closed position in accordance with an embodiment of the present invention.

FIG. 30 is a cross-sectional view of a spirometer with a flap in an open position in accordance with an embodiment of the present invention.

FIG. 31 is an exploded, perspective view of a spirometer in accordance with another embodiment of the present invention.

FIG. 32 is a perspective view of a flow chamber of a spirometer in accordance with another embodiment of the present invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

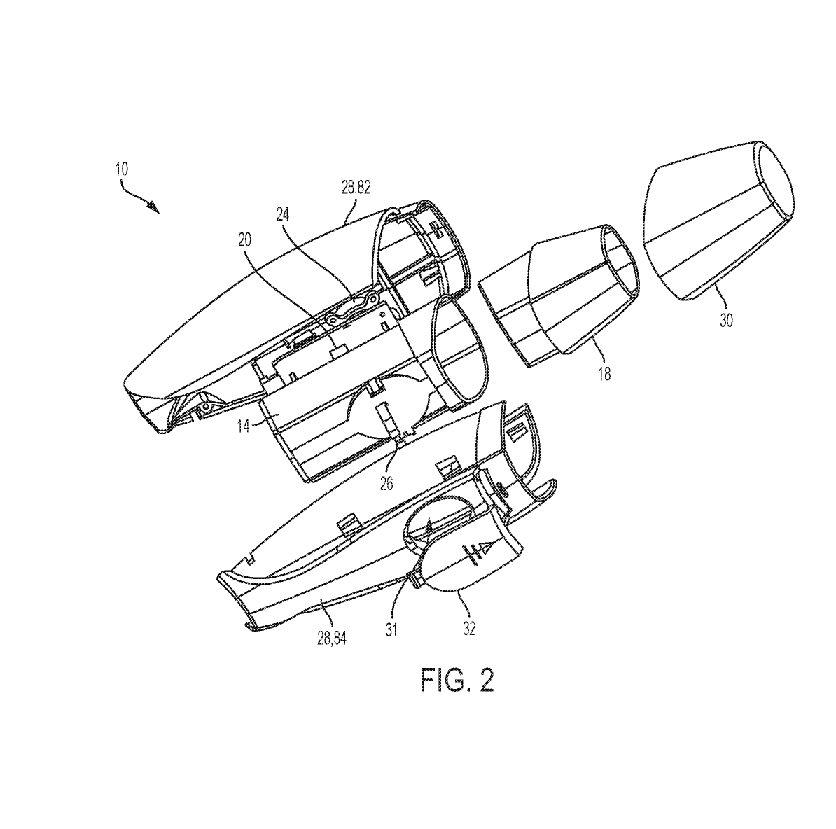

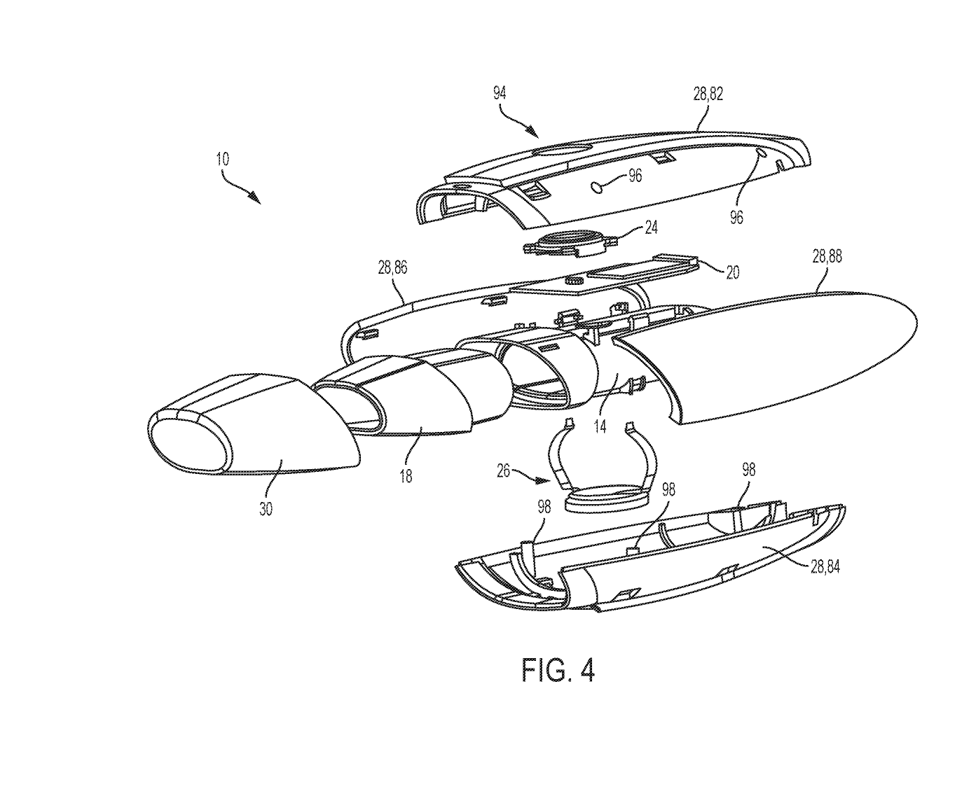

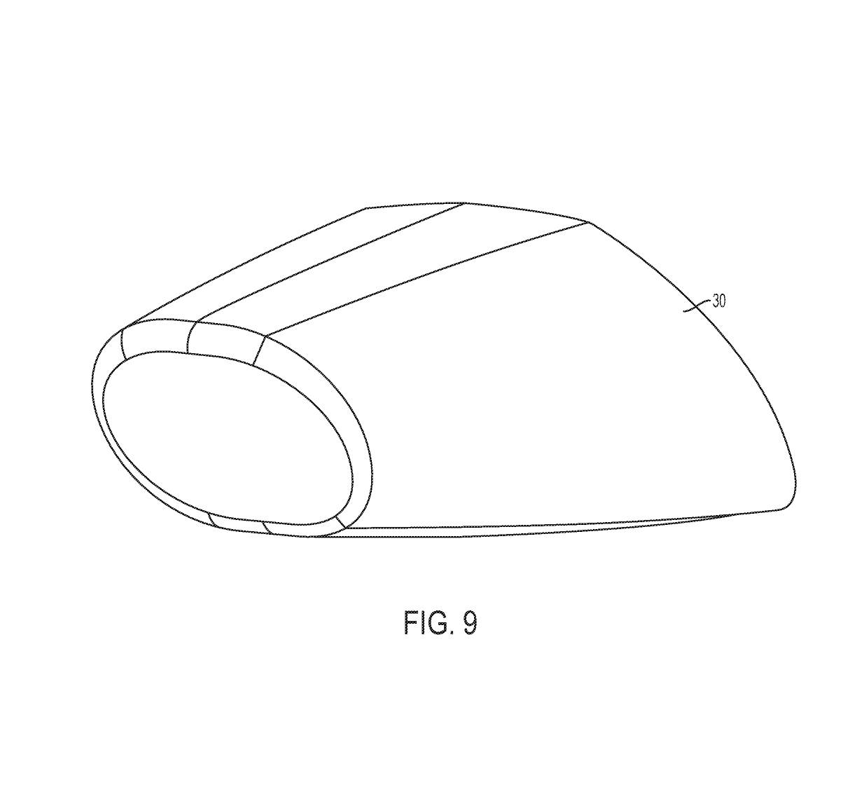

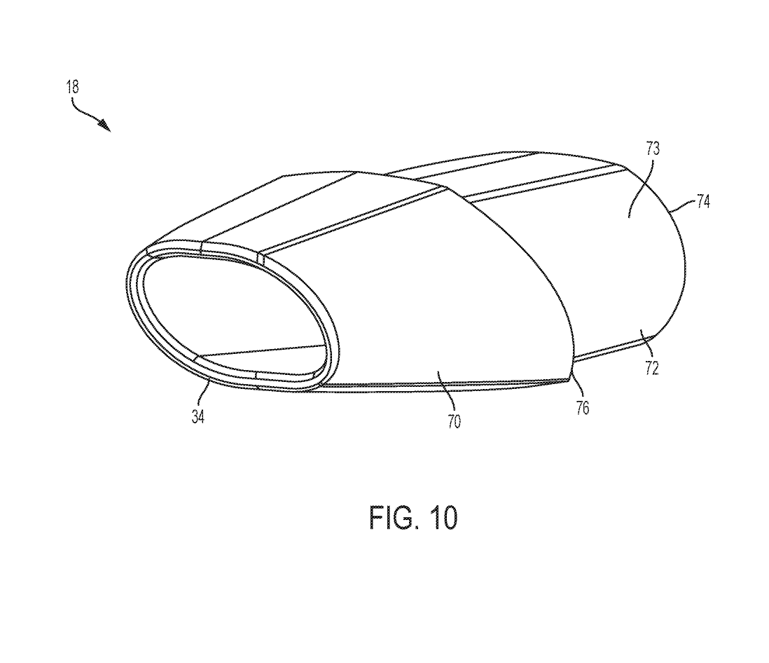

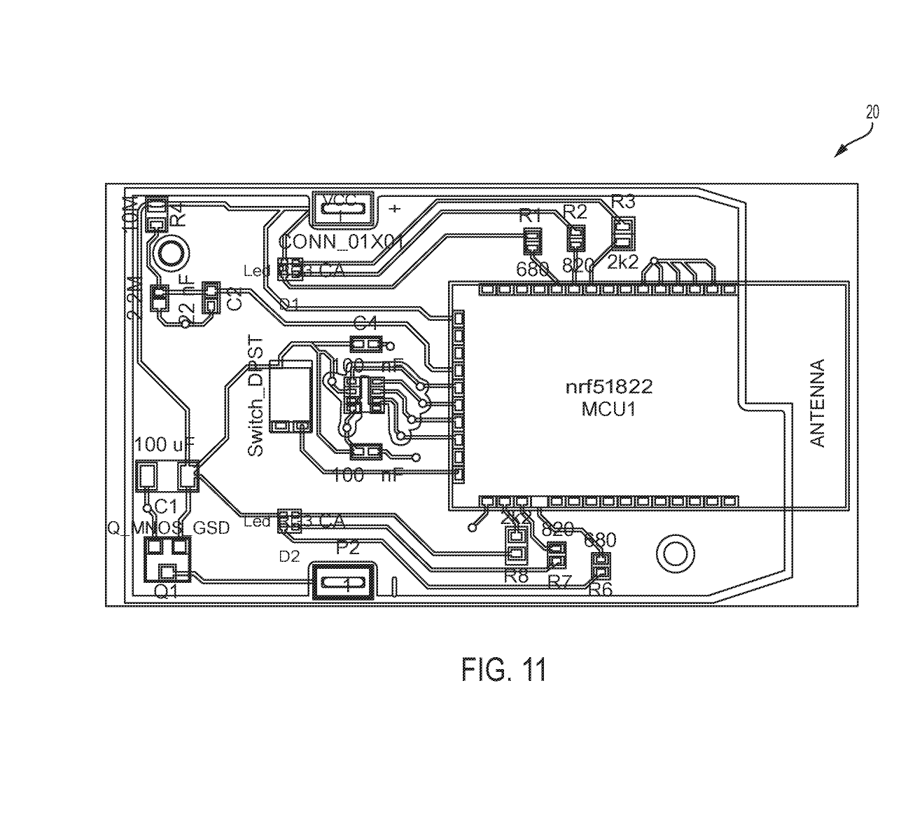

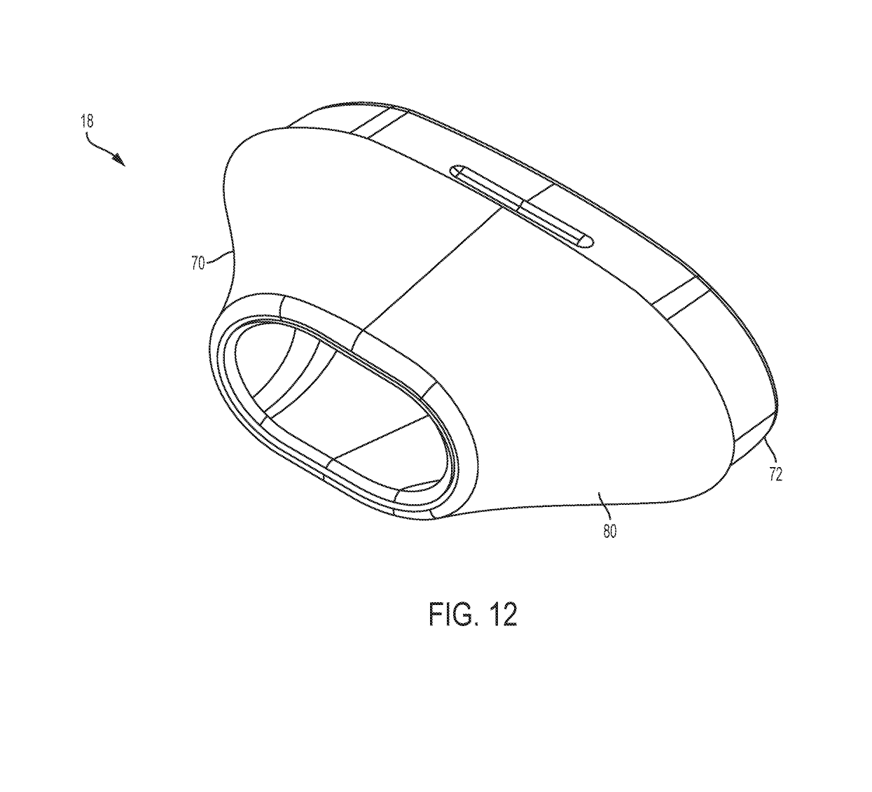

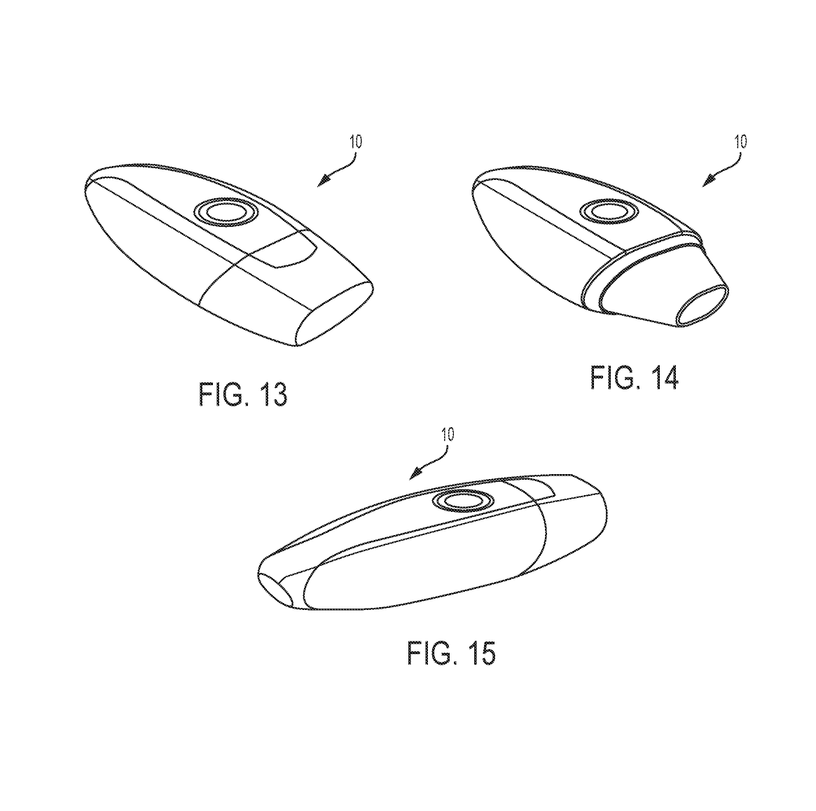

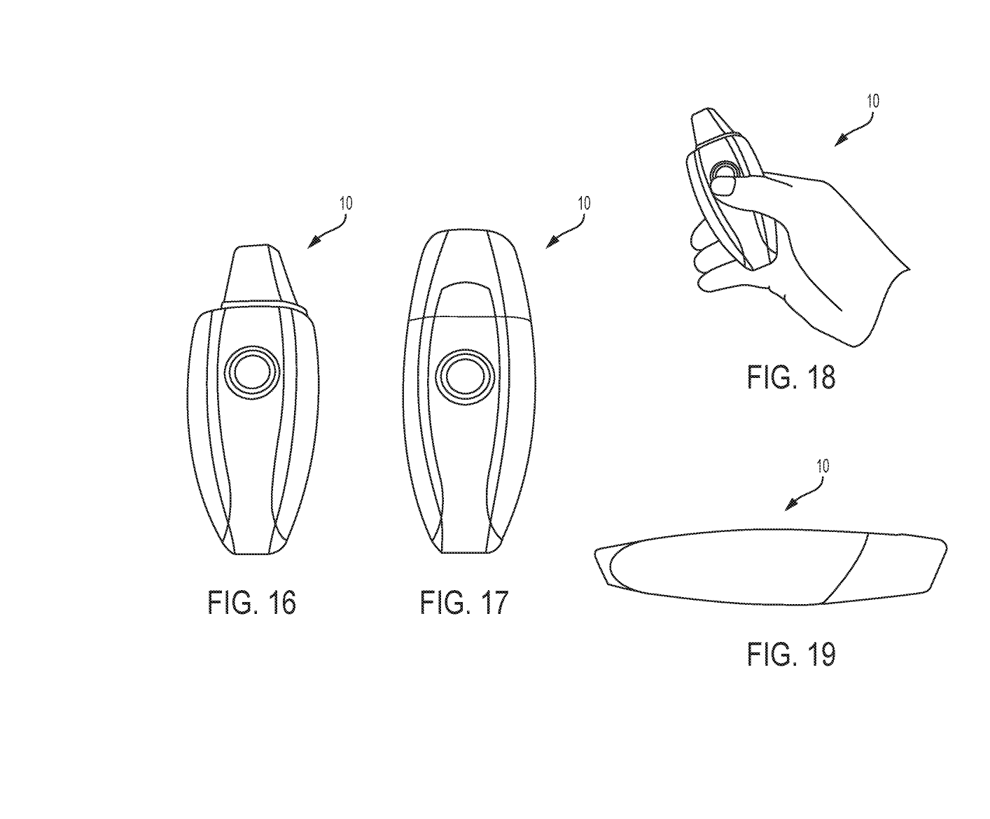

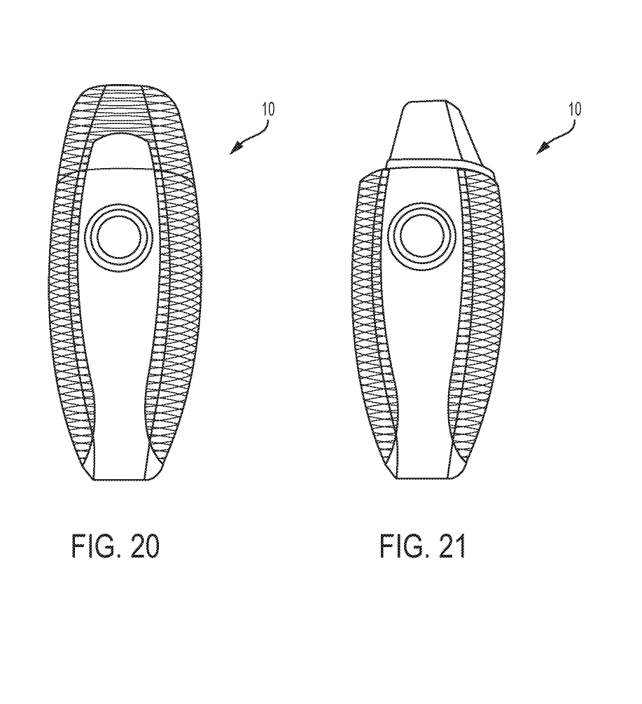

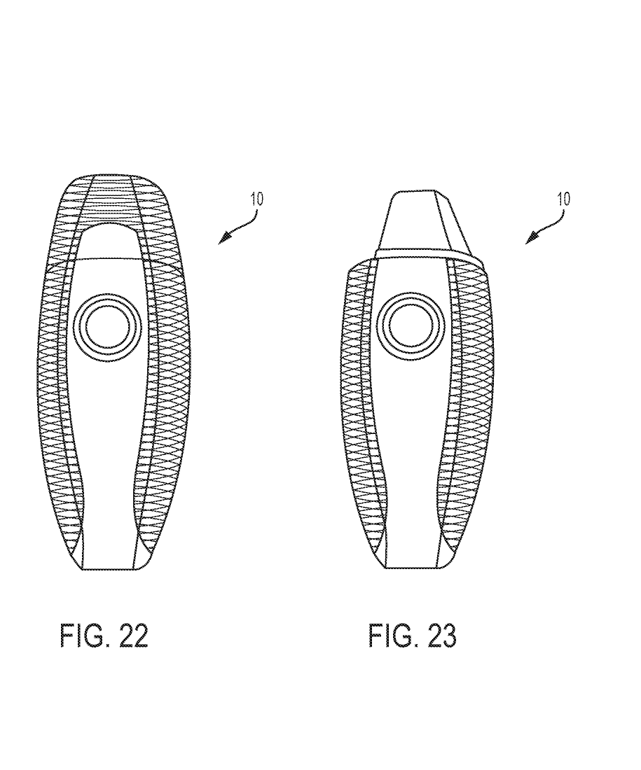

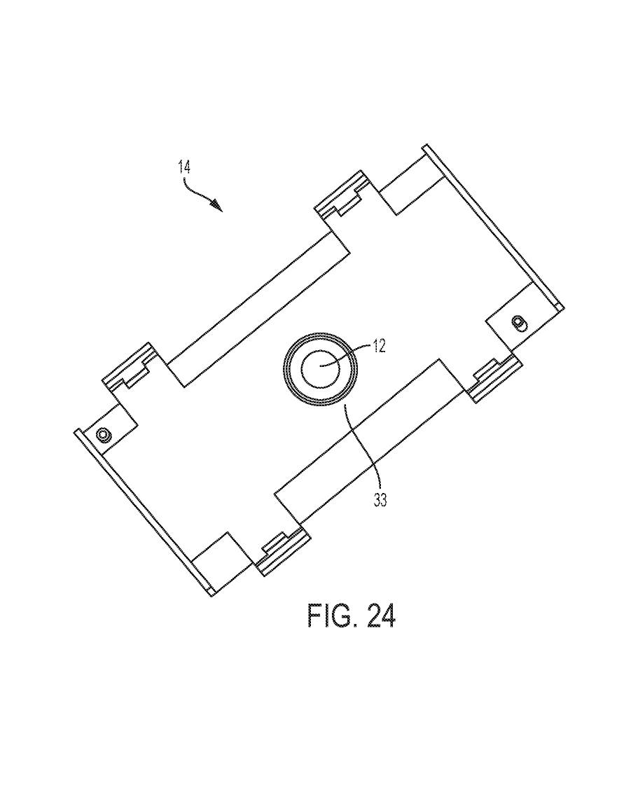

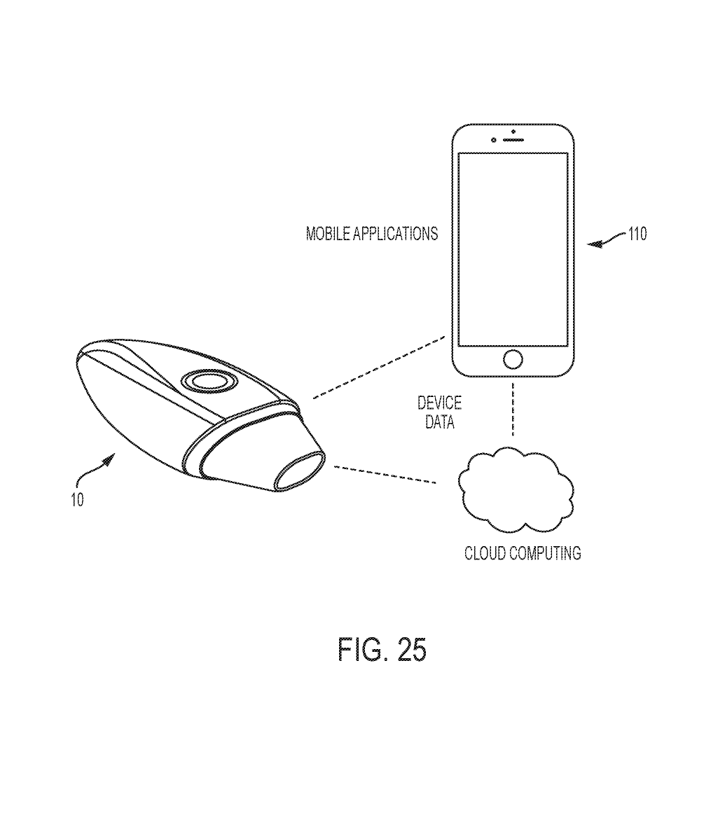

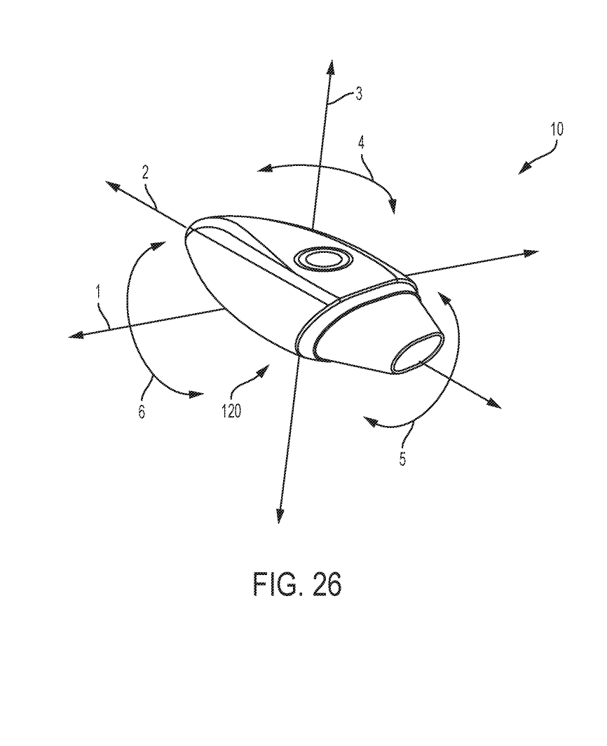

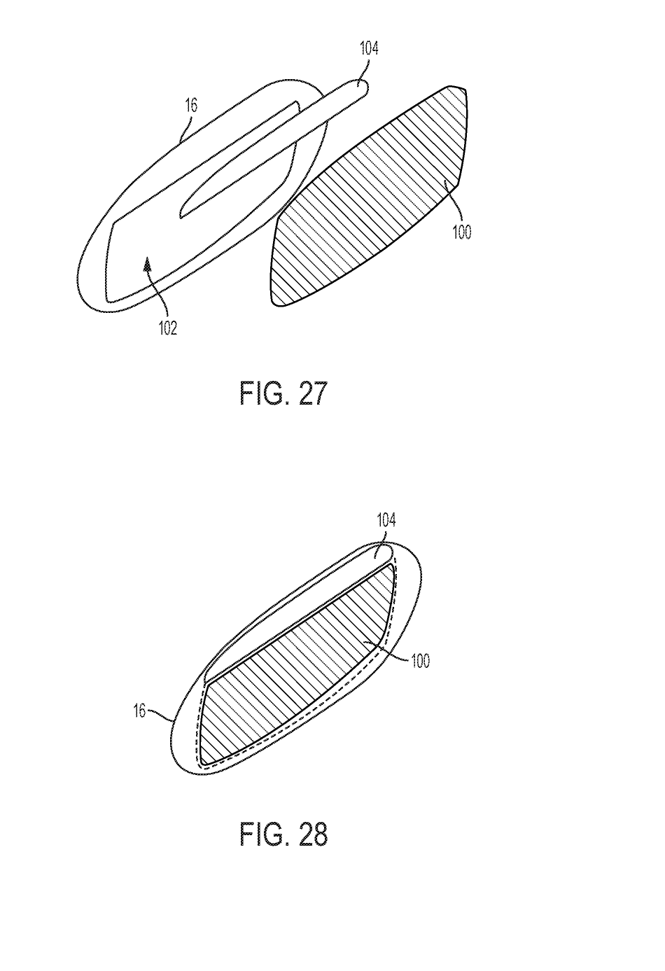

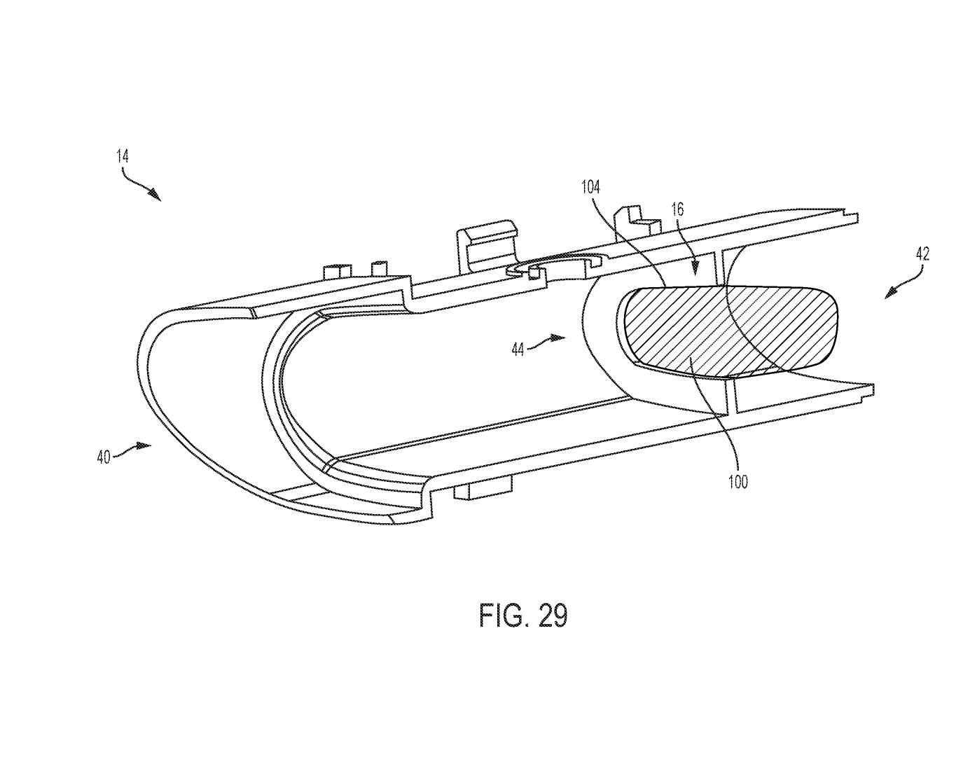

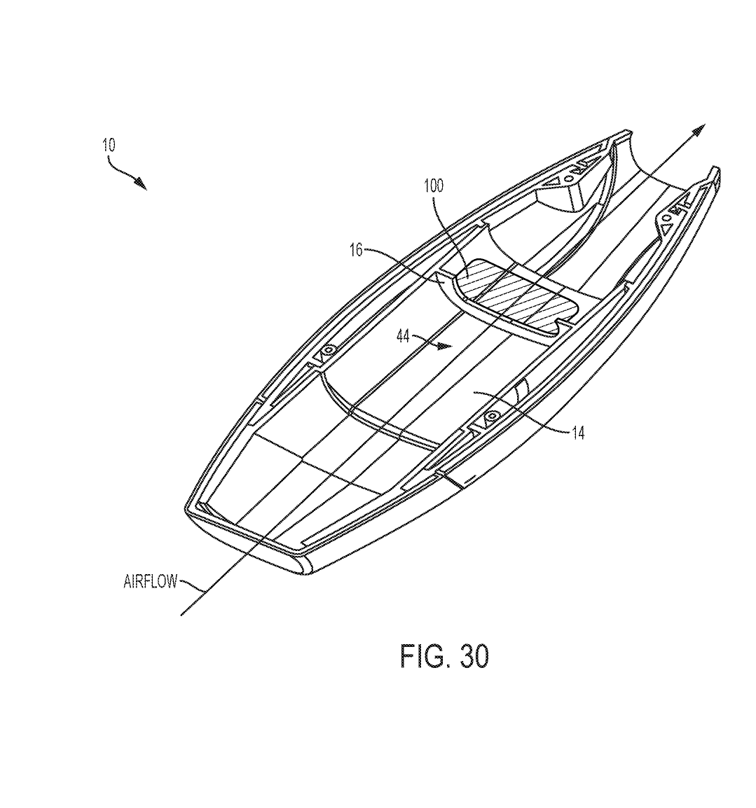

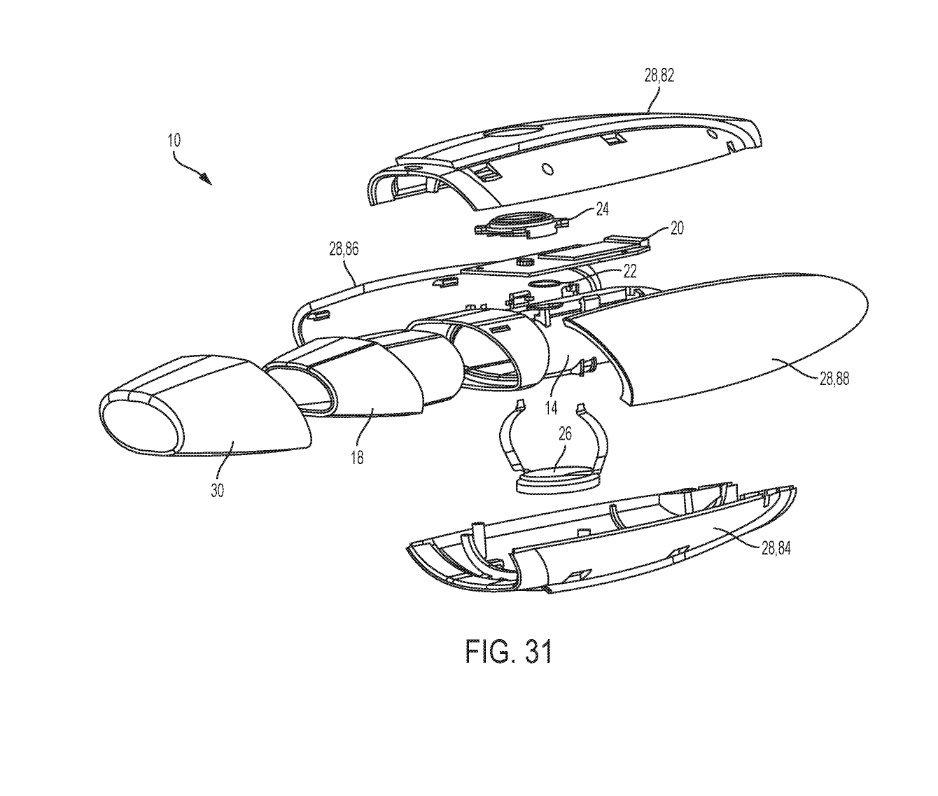

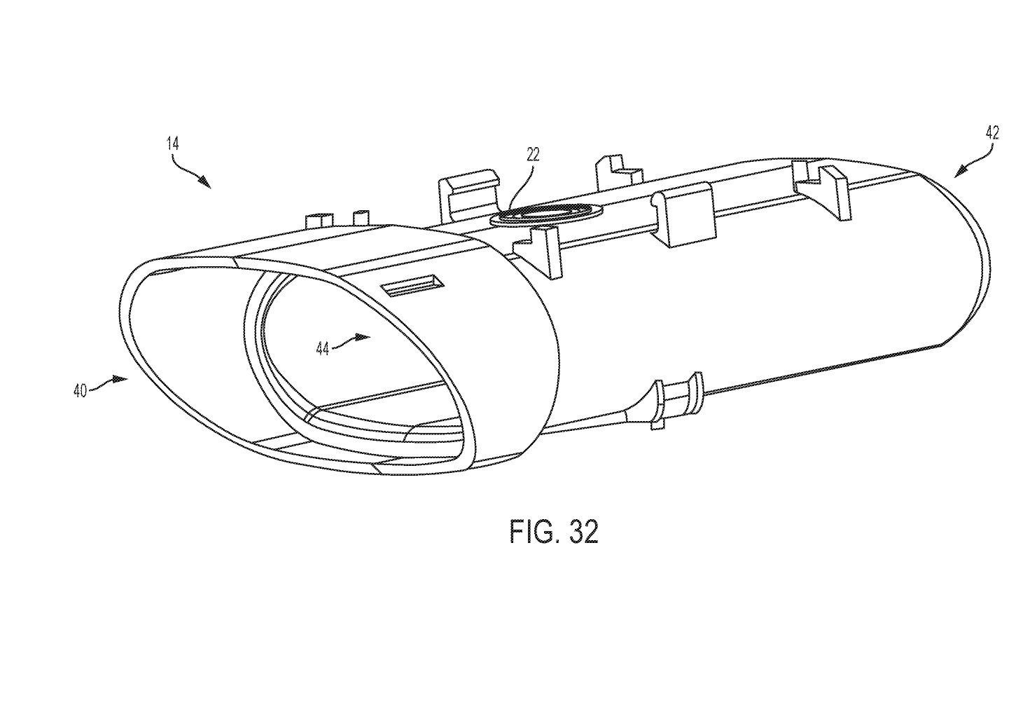

FIGS. 1-11 illustrate an exemplary embodiment of a spirometer of the present disclosure. Referring to FIGS. 1-11, a spirometer 10 of the present disclosure includes a sensor 12 and a specifically designed flow chamber 14 to create a pressure change and produce spirometry test results. In one embodiment, the sensor 12 comprises an absolute barometric pressure sensor. Examples of a suitable sensor 12 include a Bosch BME 280 sensor, a Honeywell board mount pressure sensor, or similar sensor. The spirometer 10 of the present disclosure includes a single sensor 12 positioned between an inlet of a flow chamber 14 and a barrier 16 to compute spirometry values with the pressure change. Pressure, humidity, and temperature are features that are built into the sensor 12. These sensor features and values assist in final computational spirometry test values as users complete test maneuvers.

Referring to FIGS. 1-4 and 31-32, a spirometer 10 of the present disclosure generally includes a sensor 12, a flow chamber 14, a barrier 16, a mouthpiece 18, a printed circuit board 20, a pressure seal 22, a button 24, a battery 26, a housing 28, a protective cap 30, a battery compartment 31, and a battery compartment door 32. In one embodiment, the mouthpiece 18 has a tip 34. In one embodiment, the flow chamber 14 includes a top side 36 and a bottom side 38.

Referring to FIGS. 1-8, the flow chamber 14 includes an inlet 40 and an outlet 42 and defines a fluid dynamic pathway 44 spanning the flow chamber 14. The flow chamber 14 includes a lip 46 that extends within the pathway 44 adjacent the inlet 40, an exterior wall 48, an interior wall 50, a sensor receiving orifice 52, a channel 54, securement portions 56, and a barrier or obstruction 16 defining a volumetric area. In one embodiment, the securement portions 56 can be used to secure the printed circuit board 20 to the flow chamber 14. In other embodiments, additional securement portions 56 can be used to secure the flow chamber 14 to other components of the spirometer 10 and/or to portions of the housing 28 to secure the flow chamber 14 within the housing 28 of the spirometer 10.

In one embodiment, the flow chamber 14 spans approximately the entirety of the spirometer 10. In one embodiment, the flow chamber 14 is free flowing. In one embodiment, the diameter of the flow chamber 14 is designed to allow a spirometer 10 of the present disclosure to provide readings that meet the requirements of the American Thoracic Society standards, European Respiratory Society standards, or additional national or international globally recognized regulatory bodies.

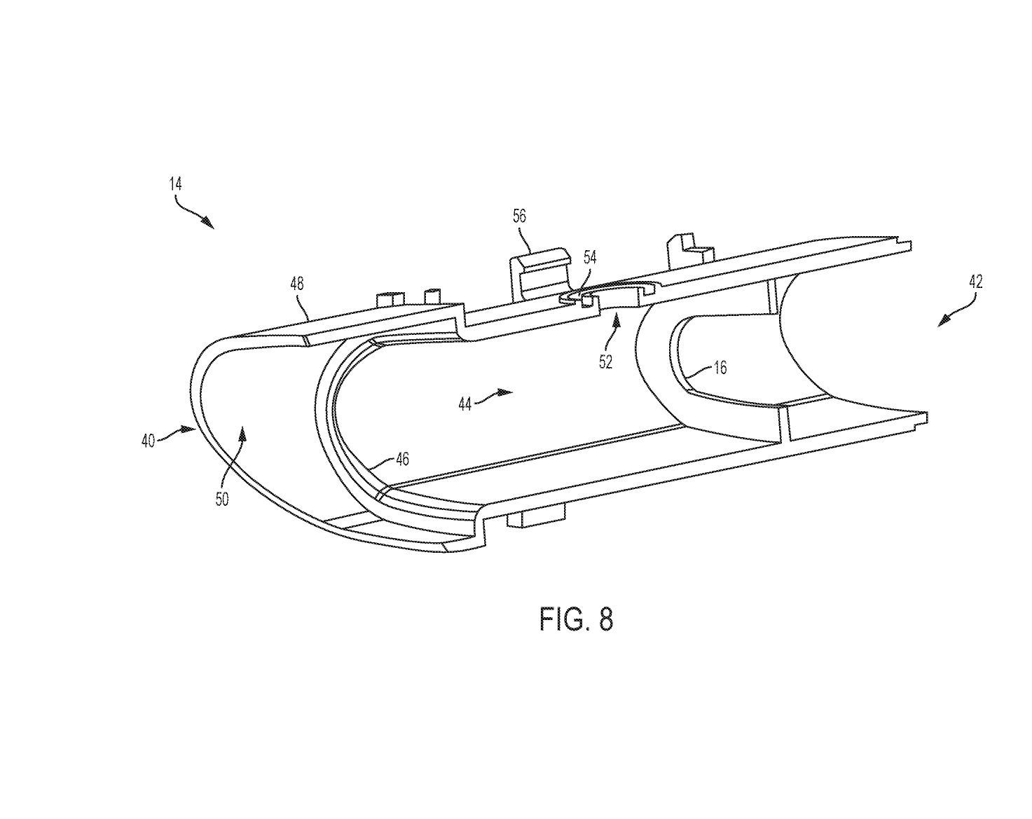

In one embodiment, the flow chamber 14 is specifically designed to create a pressure change and produce spirometry test results. Referring to FIG. 8, the flow chamber 14 includes a barrier 16 that is positioned within the flow chamber 14 to change or obstruct a portion of the pathway 44. The barrier 16 is a defined volumetric area that causes a pressure build up in the portion of the fluid dynamic pathway 44 of the flow chamber 14 between the barrier 16 and the inlet 40. In this manner, the flow chamber 14 creates a pressure change and produces a digital signal for processing final pulmonary function test results. For example, in one embodiment, when a breath of air is exhaled into the spirometer 10, the barrier 16 causes the pressure build up in the flow chamber 14 which is used to force more accurate readings of the sensor 12.

Referring to FIGS. 27-30, in one embodiment, the spirometer 10 includes a flap 100 that is movably connected to a portion of the barrier 16. The flap 100 is movable relative to the barrier 16 between a closed or initial position (FIG. 29) and an open or second position (FIG. 30).

Referring to FIG. 29, with the flap 100 in the closed position, the flap 100 covers a portion of the pathway 44 adjacent the barrier 16. For example, in one embodiment, the barrier 16 defines a barrier aperture 102 therein that is in fluid communication with the pathway 44 of the flow chamber 14. With the flap 100 in the closed position, the flap 100 covers a portion of the barrier aperture 102.

Referring to FIG. 30, with the flap 100 in the open position, the flap 100 is pivoted away from the barrier 16. For example, during use of the spirometer 10, when a breath of air is exhaled into the spirometer 10, the initial high air volume passing through the pathway 44 of the flow chamber 14 exerts a pressure on the flap 100 thereby moving the flap 100 to the open position.

However, the flow rate of air volume towards the end of exhalation tends to be lower, for example, the exhaling of approximately the last 30% of lung volume. In this case, the flap 100 functions to build up a higher pressure in the flow chamber 14, thereby allowing the lower flow rate of air volume to be analyzed properly and more efficiently.

In one embodiment, during use, the initial high air volume passing through the pathway 44 of the flow chamber 14 exerts a pressure on the flap 100 thereby moving the flap 100 to the open position. Then as the air volume decreases towards the end of exhalation, the flap 100 will move back to the closed or initial position and causes a higher pressure to be created within the flow chamber 14, thereby allowing the lower flow rate of air volume to be analyzed properly and more efficiently.

Referring to FIGS. 27-30, in one embodiment, the flap 100 is movably connected to a portion of the barrier 16. For example, in some embodiments, the flap 100 may be movably connected to a portion of the barrier 16 via a securement component 104. The securement component 104 may comprise an adhesive or other securement mechanism. In other embodiments, the flap 100 and the barrier 16 may form a unitary component. In such embodiments, the flap 100 is movably connected to the barrier 16 via a living hinge. The flap 100 and the barrier 16 may be formed via injection molding techniques. In one embodiment, the flap 100 is formed of a flexible material.

The spirometer 10 of the present disclosure includes a sensor 12. In one embodiment, the sensor 12 comprises an absolute barometric pressure sensor. Examples of a suitable sensor 12 include a Bosch BME 280 sensor, a Honeywell board mount pressure sensor, or similar sensor. The spirometer 10 of the present disclosure includes a single sensor 12 positioned between the inlet 40 of the flow chamber 14 and the barrier 16 to compute spirometry values with the pressure change. Pressure, humidity, and temperature are features that are built into the sensor 12. These sensor features and values assist in final computational spirometry test values as users complete test maneuvers.

In one embodiment, the spirometer 10 of the present disclosure includes a single sensor 12 positioned within the flow chamber 14. In one embodiment, the spirometer 10 of the present disclosure includes a single sensor 12 positioned between the inlet 40 of the flow chamber 14 and the barrier 16 to compute spirometry values with the pressure change.

In one embodiment, the single sensor 12 is located at a central location from the sides of the spirometer 10. In one embodiment, the single sensor 12 is located at a top side 36 of the flow chamber 14 to avoid contact with moisture. In other embodiments, the single sensor 12 may be located at other positions, e.g., a bottom side 38 of the flow chamber 14, with a membrane or humidity protection cover disposed over a sensor 12 to avoid direct or indirect contact with moisture.

The sensor 12 of the present disclosure is protected from the 100% relative humidity being expelled by an individual as a test is completed. This may induce a significant amount of sensor noise and as testing procedures are repeated back to back, humidity can cause significant degradation in data accuracy. For these reasons, referring to FIG. 24, in one embodiment, a spirometer 10 of the present disclosure includes a loosely fit membrane 33 that is able to move in and out of the hole, e.g., a sensor receiving orifice 52, where the sensor 12 sits. The membrane 33 protects the sensor 12 from humidity but still allows for the pressure change to occur as exhalation is made through the device. In one embodiment, the membrane 33 protectively covers the sensor 12 and the membrane 33 is movable relative to a portion of the flow chamber 14. For example, in some embodiments, the membrane 33 is configured to be “tented” or flexible in nature to allow room for movement. In such a configuration, this flexibility accommodates for pressure changes as the fluid dynamics change. In other embodiments, the membrane 33 can sit directly against a portion of the flow chamber 14. In one embodiment, the membrane 33 is hydrophobic, breathable, and flexible to allow for pressure change.

In one embodiment, the single sensor 12 is located at a point between approximately 10%-90% of the distance from the tip 34 of the mouthpiece 18 to the barrier 16. In one embodiment, the single sensor 12 is located at a point between approximately 15%-85% of the distance from the tip 34 of the mouthpiece 18 to the barrier 16. In one embodiment, the single sensor 12 is located at a point between approximately 20%-80% of the distance from the tip 34 of the mouthpiece 18 to the barrier 16. In one embodiment, the single sensor 12 is located at a point at least 20% away from the tip 34 of the mouthpiece 18. In one embodiment, the single sensor 12 is located at a point between approximately 25%-75% of the distance from the tip 34 of the mouthpiece 18 to the barrier 16. For example, in one embodiment, the single sensor 12 is located at a point at least 25% away from the tip 34 of the mouthpiece 18 and at least 25% away from the barrier 16.

In one embodiment, the single sensor 12 is located at a point between approximately 60%-80% of the distance from the tip 34 of the mouthpiece 18 to the barrier 16. In one embodiment, the single sensor 12 is located at a point between approximately 75%-80% of the distance from the tip 34 of the mouthpiece 18 to the barrier 16. In one embodiment, the single sensor 12 is located at a point between approximately 79% of the distance from the tip 34 of the mouthpiece 18 to the barrier 16.

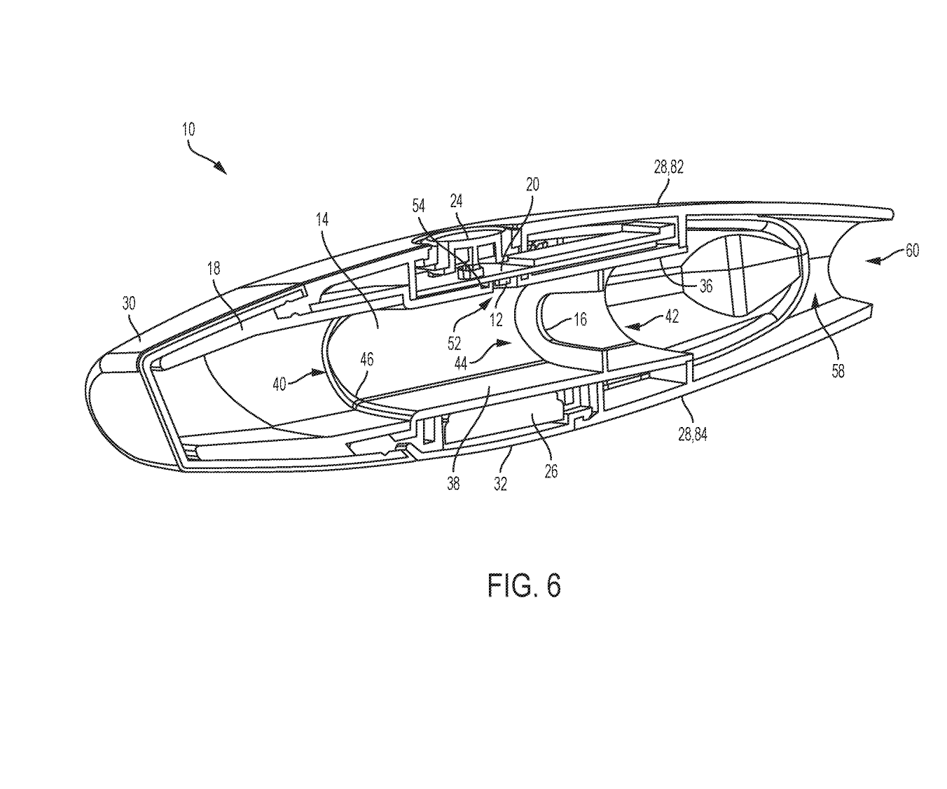

Referring to FIGS. 6-8, the flow chamber 14 defines a sensor receiving orifice 52. In one embodiment, the sensor receiving orifice 52 is positioned between the inlet 40 and the barrier 16. The sensor 12 is securably receivable within the sensor receiving orifice 52. In this manner, the sensor 12 is positioned between the inlet 40 of the flow chamber 14 and the barrier 16 to compute spirometry values. This position of the sensor 12 relative to the pathway 44 of the flow chamber 14 and the barrier 16 is important so that the sensor 12 is positioned to allow the fluid dynamics of the spirometer 10 to be properly aligned for adequate fluid dynamic pressure balancing.

The sensor 12 has a first height and the sensor receiving orifice 52 has a second height. In one embodiment, the second height of the sensor receiving orifice 52 is greater than the first height of the sensor 12. In this manner, referring to FIG. 6, with the sensor 12 securely received and positioned within the sensor receiving orifice 52, no portion of the sensor 12 extends beyond a bottom portion of the sensor receiving orifice 52 into the pathway 44 of the flow chamber 14. This allows the sensor 12 to be protectively positioned within the sensor receiving orifice 52. This prevents and/or reduces any moisture from contacting the sensor 12 during use of the spirometer 10. In one embodiment, a membrane 33 (FIG. 24) may also be positioned over the sensor 12 to protectively cover the sensor 12 and prevent humidity from directly or indirectly inducing sensor signal noise.

Referring to FIGS. 6-8 and 31-32, the flow chamber 14 also defines a channel 54 around the periphery of the sensor receiving orifice 52. In one embodiment, the channel 54 receives a pressure seal 22. In one embodiment, the pressure seal 22 is an O-ring. In such embodiments, the O-ring is positioned between the flow chamber 14 and the printed circuit board 20 and protectively seals the sensor 12 and the printed circuit board 20. In this manner, the O-ring prevents moisture from contacting the printed circuit board 20 during use of the spirometer 10.

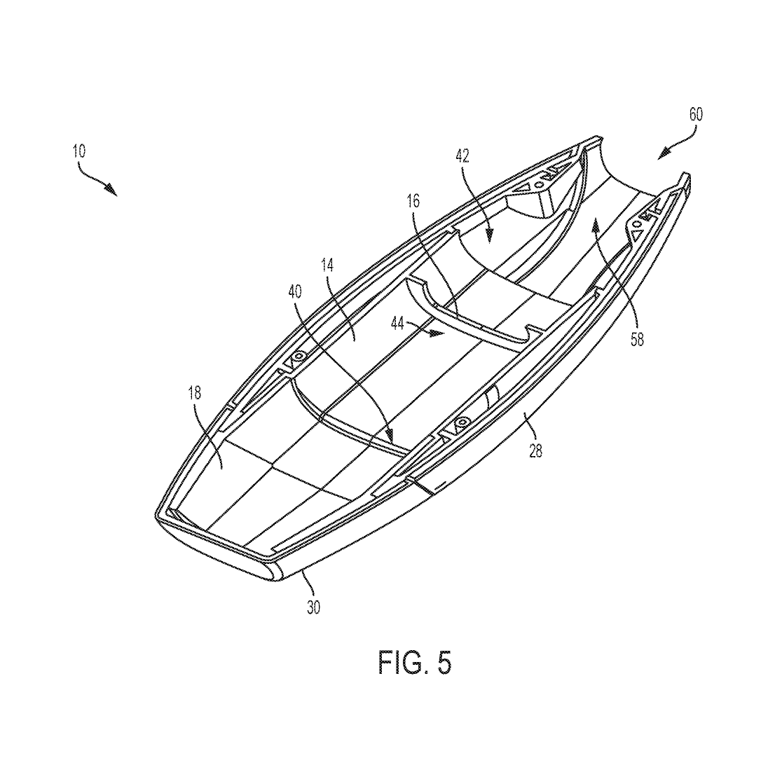

Referring to FIG. 5, in one embodiment, the spirometer 10 includes an exit chamber 58. The exit chamber 58 is defined between a rear portion 60 of the spirometer 10 and the outlet 42 of the flow chamber 14.

Referring to FIGS. 1-4, 6, and 10, the spirometer 10 includes a mouthpiece 18. In one embodiment, the mouthpiece 18 includes a first portion 70 and a second portion 72. The second portion 72 includes an exterior wall 73 and an end wall 74. In one embodiment, the first portion 70 of the mouthpiece 18 has a cross-section that has a greater area than a cross-section of the second portion 72 of the mouthpiece 18 such that a shoulder 76 is defined therebetween. In one embodiment, the mouthpiece 18 is removably connectable with the flow chamber 14.

The mouthpiece 18 securely fits in the inlet 40 of the flow chamber 14 as shown in FIG. 6. For example, the second portion 72 of the mouthpiece 18 securely fits in the inlet 40 of the flow chamber 14 such that the exterior wall 73 of the second portion 72 is in contact with the interior wall 50 of the inlet 40 of the flow chamber 14 as shown in FIG. 6. Also, the second portion 72 of the mouthpiece 18 securely fits in the inlet 40 of the flow chamber 14 such that the end wall 74 of the second portion 72 is in contact with the lip 46 of the flow chamber 14. In this manner, with the mouthpiece 18 received within the inlet 40 of the flow chamber 14, there are no gaps disrupting fluid dynamics between the mouthpiece 18 and the flow chamber 14.

By having no gaps between the mouthpiece 18 and the flow chamber 14, a spirometer 10 of the present disclosure prevents having any spaces where pressure could be lost within the flow chamber 14 of the spirometer 10 which may increase or decrease the efficiency of the device.

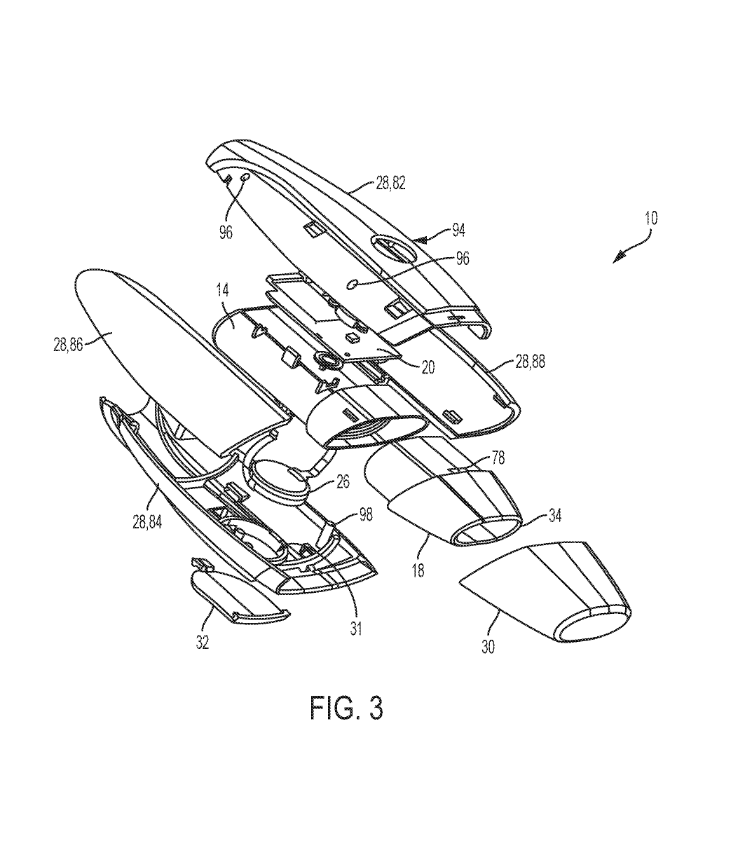

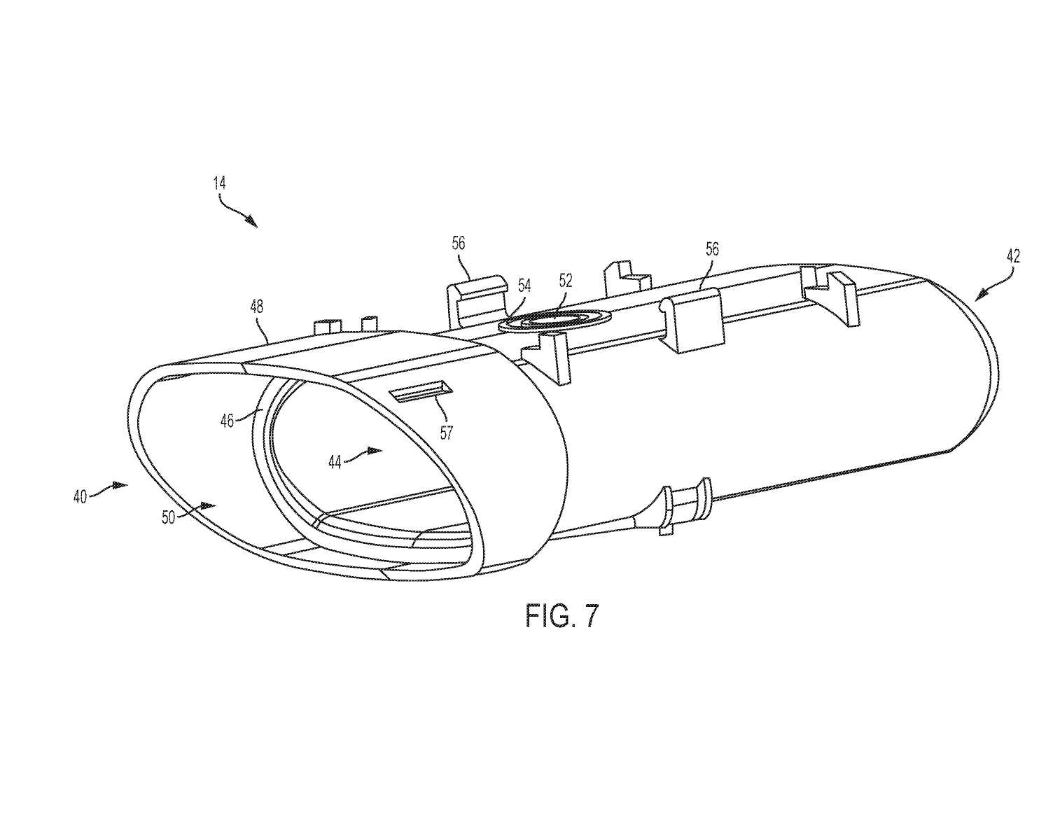

The mouthpiece 18 securely fits in the inlet 40 of the flow chamber 14 as shown in FIG. 6. In one embodiment, a mechanical locking mechanism is included to provide secure engagement between the mouthpiece 18 and the flow chamber 14. In one embodiment, a snap-fit engagement is provided between the mouthpiece 18 and the flow chamber 14. For example, the mouthpiece 18 may include a mechanical locking tab 78 (FIG. 3) that may be securely received within a mechanical locking slot 57 (FIG. 7) of the flow chamber 14.

Referring to FIG. 12, in one embodiment, the mouthpiece 18 may include a tapered design. For example, referring to FIG. 12, the first portion 70 of the mouthpiece 18 may include a tapered portion or curved side surfaces 80. By having the sides of the mouthpiece 18 curved, the mouthpiece 18 is easier for a child to comfortably fit the mouthpiece 18 within their mouth during use of the spirometer 10. Additionally, by having the sides of the mouthpiece 18 curved, the mouthpiece 18 allows for any range of patient age and/or size to properly conduct spirometry maneuvers using the spirometer 10 of the present disclosure.

In one embodiment, the spirometer 10 may include a disposable mouthpiece sheath that protectively covers the mouthpiece 18 during use of the spirometer 10. In use of the spirometer 10, a mouthpiece sheath may be securely positioned over the first portion 70 of the mouthpiece 18. In this manner, any contact between the mouth of a user and the spirometer 10 is only via the sterile mouthpiece sheath thereby preventing undesirables from contacting the mouthpiece 18. After use, the mouthpiece sheath can be removed from the mouthpiece 18 and thrown out. For a subsequent use of the spirometer 10, a new sterile mouthpiece sheath can be securely positioned over the first portion 70 of the mouthpiece 18. In this manner, contamination of the mouthpiece 18 is prevented for future uses and/or by different users.

Referring to FIGS. 1-4 and 11, the spirometer 10 includes a printed circuit board 20 that is in communication with the sensor 12 or secondary devices. The printed circuit board 20 mechanically supports and electrically connects the components of the spirometer 10. In one embodiment, the spirometer 10 may include a 100 uF capacitor, a 0.022 uF capacitor, a 0.1 uF capacitor, a RGB LED, contacts, an open jumper, a MCU/BLE module, an N-channel MOSFET, a 680 ohm resistor, a 820 ohm resistor, a 2200 ohm resistor, a 7.5 Mohm resistor, a 2.2 Mohm resistor, a pressure sensor, and a momentary push button, e.g., a button 24 that is in communication with the printed circuit board 20. In one embodiment, the button 24 is transitionable between an on position and an off position. Actuation of the button 24 can provide several functions of the spirometer 10, such as powering the device on and off, taking a reading, bringing the device back from sleep mode, and a variety of other programmable functions.

Referring to FIGS. 1-4, the spirometer 10 includes a battery 26 that can be securely received within a battery compartment 31. In one embodiment, access to the battery compartment 31 can be provided by removing a mechanically attached battery compartment door 32 that is removably connectable to a portion of the housing 28. In one embodiment, the battery 26 comprises a coin cell battery.

Referring to FIGS. 1-4, the spirometer 10 includes a housing 28 that protectively covers the internal components of the spirometer 10, e.g., the flow chamber 14, the sensor 12, the printed circuit board 20, and the battery 26. In one embodiment, the housing 28 includes a top housing 82, a bottom housing 84, a first side housing 86, and a second side housing 88. The housing components can be securely connected theretogether via a snap-fit engagement including a plurality of mechanical locking tabs on a first housing component that are securely received within respective mechanical locking slots on an opposite second housing component. In other embodiments, other securement mechanisms between the housing components can be utilized.

In one embodiment, with the housing components assembled together, four fasteners can be used to further secure the housing components theretogether. For example, in one embodiment, the top housing 82 includes four fastener holes 96 and the bottom housing 84 includes four fastener receiving posts 98. With the housing components properly assembled together, four fasteners can be secured to the housing components by being received within the respective fastener holes 96 and locked to the respective fastener receiving posts 98.

Referring to FIG. 1, the top housing 82 defines a button receiving aperture 94. In one embodiment, with the spirometer 10 assembled, a portion of the button 24 is contained within the button receiving aperture 94 of the top housing 82. In this manner, with the spirometer 10 assembled, a portion of the button 24 is accessible by a user so that a user can transition the button 24 between an on position and an off position and/or mechanical or electrical states.

Referring to FIGS. 1-4 and 9, the spirometer 10 includes a cap 30 that protectively covers the mouthpiece 18. The cap 30 is removably connectable to a portion of the housing 28 and/or the mouthpiece 18. Referring to FIG. 6, in one embodiment, a mechanical fit is provided between the cap 30 and the housing 28 and/or the mouthpiece 18.

Referring to FIGS. 13-26, a variety of the external features and/or ergonomics of a spirometer 10 of the present disclosure are illustrated. In one embodiment, the external features and/or housing components of the spirometer 10 may be injection molded with plastics that allow for UV sensitization.

How the sensor 12 of the spirometer 10 of the present disclosure interacts with the electronics of the device will now be discussed. A spirometer 10 of the present disclosure uses one absolute barometric pressure sensor positioned within a flow chamber 14 between the inlet 40 and the barrier 16. As airflow passes through the pathway 44 of the flow chamber 14 past the sensor 12, the pathway 44 of the flow chamber 14 experiences a change in pressure. Signal processing from the sensor 12 occurs within the firmware that lives on the printed circuit board and microprocessor of the device itself. The processed signal data from the sensor 12 is used in calculus to determine final pulmonary function values based on pressure changes inside of the pathway 44 of the flow chamber 14. In one embodiment, an Arduino based microcontroller connects the sensor 12, Bluetooth, and/or other desired devices, and allows for basic data input/output, and iOS, Android, or Desktop computer connectivity via Bluetooth or similar technology. As a user conducts spirometry maneuvers, some light computational work takes place on the MCU and the final test results may be calculated off device on a separate medium.

How data is pulled to compile readings will now be discussed. As a spirometry maneuver is completed the needed values for calculation are transmitted to a means of interpretation via Bluetooth. Secondary software off the device itself such as an Android, iOS, or Desktop application can be used to review the data from the spirometer 10. No pulmonary function data is stored on the printed circuit board or on the hardware itself. It is purely a means of transmission and computational values are concluded on secondary hardware such as iOS, Android, or Desktop computers.

In one embodiment, a spirometer 10 of the present disclosure is compatible with backend software support and integrations for Apple Healthkit, Google Fit, and a variety of top electronic medical record or insurance provider software providers. These software integrations include, but are not limited to, United Health, American Family Ventures, or other insurance providers, eClinicalWorks, McKesson, Cerner, Athena Health, and Allscripts. This applies to any API application programming interface provided for electronic medical record integration or insurance provider integration.

Advantageously, a spirometer having an absolute barometric pressure sensor of the present disclosure eliminates the requirement of calibration associated with conventional spirometers. Conventional spirometers require the time consuming and expensive step of calibration using a three (3) liter syringe to push air through a spirometer to test for calibration before using the spirometer. By eliminating this calibration step, a spirometer of the present disclosure makes the testing procedure, along with training for new healthcare professionals, very simple. They can simply open the packaging, unbox the unit, power it on, and begin testing. Previously, conventional spirometers required sensor calibration to detect minute changes in deviation on sensor resolution. Due to the accuracy of a spirometer having an absolute barometric pressure sensor of the present disclosure, the time consuming and expensive step of calibration is eliminated.

Furthermore, a spirometer having an absolute barometric pressure sensor of the present disclosure provides for automatic sensor recalibration between tests. For example, after a user completes a test, a spirometer of the present disclosure can automatically be zeroed out again. In one embodiment, this may be based on the altitude and environmental conditions. For example, relative humidity, altitude, pressure and temperature conditions can be taken into account and incorporated into the calculus to determine final output values.

Also, a spirometer having an absolute barometric pressure sensor of the present disclosure provides for automatic sensor recalibration on startup. For example, if the device is restarted at any time, the spirometer is always automatically recalibrated and zeroed out.

Furthermore, a spirometer having an absolute barometric pressure sensor of the present disclosure provides for automatic sensor recalibration on any device faults. For example, if the device locks up, the spirometer is automatically rebooted, recalibrated, and zeroed out.

Referring to FIG. 25, a spirometer 10 of the present disclosure can be in communication with a mobile device 110. Advantageously, a spirometer 10 of the present disclosure provides interpolation between sensors on a mobile device 110 and sensors on a spirometer 10. For example, by incorporating pressure sensor data available on a mobile device 110 used for testing and sensors within the spirometer 10 itself, secondary validation of pressure and zeroing out is provided. Thus, if one sensor is reading one value range and another sensor is reading another value range, interpolation between the two occurs to clean up the baseline data. In one embodiment, the sensor of the spirometer 10 comprises an absolute barometric pressure sensor.

In one embodiment, a mobile device 110 is in communication with the spirometer 10 and the mobile device 110 includes a secondary sensor. The secondary sensor of the mobile device 110 improves accuracy in the calculus to determine final pulmonary function values based on pressure changes inside of the spirometer 10. With multiple environmental sensors present, the pressure information, temperature and any other environmental data can be incorporated into these final values.

In one embodiment, the mobile device 110 being in communication with the spirometer 10 allows for data exchange and/or communication between the spirometer 10 and the mobile device 110. For example, the mobile device 110 being in communication with the spirometer 10 allows for data exchange and communication between environmental sensors of the mobile device 110, the hardware sensors of the spirometer 10, and the value of use in computing predicted values.

Another advantage of the mobile device 110 being in communication with the spirometer 10 is that error detection of faulty hardware is provided. For example, determination of a damaged or poorly calibrated spirometer 10 can be identified by the referenced interpolation between environmental sensors on a mobile device 110 and sensors inside the spirometer 10.

In one embodiment, a reference point for a spirometer's level of degradation or degree to which it is damaged is based on determining the percentage of mean difference between sensor values on the mobile device 110 and the spirometer 10. In one embodiment, if a degree of accuracy of the spirometer 10 exceeds 10-15% from the sensors of a mobile device 110, the spirometer 10 can be flagged for potential damage or poor calibration. In one embodiment, in such a case, the spirometer 10 can be invalidated and determined not fit for use.

Referring to FIG. 26, in one embodiment, a spirometer 10 of the present disclosure may also incorporate an accelerometer 120. In such an embodiment, the data from the accelerometer 120 is used to detect movement during a test and assist in determining the level of potential error that may have occurred. For example, if a user moves too much, the user may be informed of this and that the test was probably not completed accurately due to the excessive movement. In one embodiment, as someone exhales, the information obtained from the accelerometer 120 can be used along with defined acceptable parameters for the patient's movement. If a patient's movement meets or exceeds that of predefined acceptable parameters, a test can be additionally validated or invalidated. In one exemplary embodiment, a spirometer 10 includes an accelerometer 120, for example, a three axis accelerometer, gyro or IMU inertial measurement unit. For example, the accelerometer 120 could be an Invensense—gyroscope, mems, triple axis, qfn-24-ITG-3200 unit.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A spirometer, comprising:
a flow chamber defining a pathway, the flow chamber having an inlet and an outlet;
a barrier defining a volumetric area, the barrier positioned within the flow chamber to obstruct a portion of the pathway; and
a sensor positioned between the inlet and the barrier.

2. The spirometer of claim 1, wherein the sensor comprises an absolute barometric pressure sensor.

3. The spirometer of claim 1, wherein the flow chamber includes a lip extending within the pathway adjacent the inlet.

4. The spirometer of claim 3, further comprising a mouthpiece having a first portion and a second portion, the second portion having an end wall, the second portion of the mouthpiece received within the inlet of the flow chamber such that the end wall contacts the lip of the flow chamber.

5. The spirometer of claim 4, wherein, with the mouthpiece received within the inlet of the flow chamber, there are no gaps between the mouthpiece and the flow chamber.

6. The spirometer of claim 4, further comprising a disposable mouthpiece sheath that protectively covers the mouthpiece.

7. The spirometer of claim 4, wherein the mouthpiece includes a tapered portion.

8. The spirometer of claim 4, further comprising a printed circuit board in communication with the sensor.

9. The spirometer of claim 8, further comprising a pressure seal between the flow chamber and the printed circuit board.

10. The spirometer of claim 8, further comprising a button in communication with the printed circuit board, the button transitionable between an on position and an off position.

11. The spirometer of claim 10, further comprising a battery.

12. The spirometer of claim 11, further comprising a housing that protectively covers the flow chamber, the sensor, the printed circuit board, and the battery.

13. The spirometer of claim 12, wherein the housing comprises a top housing, a bottom housing, a first side housing, and a second side housing.

14. The spirometer of claim 12, further comprising a cap that protectively covers the mouthpiece, the cap removably connectable to a portion of the housing.

15. A spirometer, comprising:
a flow chamber defining a pathway, the flow chamber having an inlet, an outlet, and a lip extending within the pathway adjacent the inlet;
a barrier defining a volumetric area, the barrier positioned within the flow chamber to obstruct a portion of the pathway;
a sensor positioned between the inlet and the barrier, wherein the sensor comprises an absolute barometric pressure sensor, and
a mouthpiece having a first portion and a second portion, the second portion having an end wall, the second portion of the mouthpiece received within the inlet of the flow chamber such that the end wall contacts the lip of the flow chamber.

16. The spirometer of claim 15, wherein, with the mouthpiece received within the inlet of the flow chamber, there are no gaps between the mouthpiece and the flow chamber.

17. The spirometer of claim 15, further comprising a disposable mouthpiece sheath that protectively covers the mouthpiece.

18. The spirometer of claim 15, wherein the mouthpiece includes a tapered portion.

19. The spirometer of claim 15, further comprising a printed circuit board in communication with the sensor.

20. The spirometer of claim 19, further comprising a pressure seal between the flow chamber and the printed circuit board.

21. The spirometer of claim 19, further comprising a button in communication with the printed circuit board, the button transitionable between an on position and an off position.

22. The spirometer of claim 21, further comprising a battery.

23. The spirometer of claim 22, further comprising a housing that protectively covers the flow chamber, the sensor, the printed circuit board, and the battery.

24. The spirometer of claim 23, wherein the housing comprises a top housing, a bottom housing, a first side housing, and a second side housing.

25. The spirometer of claim 23, further comprising a cap that protectively covers the mouthpiece, the cap removably connectable to a portion of the housing.

26. A spirometer, comprising:
a flow chamber defining a pathway, the flow chamber having an inlet and an outlet;
a barrier defining a volumetric area, the barrier positioned within the flow chamber to obstruct a portion of the pathway;
a sensor positioned between the inlet and the barrier; and
a membrane that protectively covers the sensor, the membrane movable relative to a portion of the flow chamber.

27. The spirometer of claim 26, wherein the sensor comprises an absolute barometric pressure sensor.

28. In combination:
a spirometer, comprising:
a flow chamber defining a pathway, the flow chamber having an inlet and an outlet;
a barrier defining a volumetric area, the barrier positioned within the flow chamber to obstruct a portion of the pathway; and
a sensor positioned between the inlet and the barrier; and
a mobile device in communication with the spirometer, the mobile device having a secondary sensor.

29. The combination of claim 28, wherein the sensor comprises an absolute barometric pressure sensor.

30. A spirometer, comprising:
a flow chamber defining a pathway, the flow chamber having an inlet and an outlet;
a barrier defining a volumetric area, the barrier positioned within the flow chamber to obstruct a portion of the pathway;
a sensor positioned between the inlet and the barrier; and
an accelerometer that detects movement.

31. The spirometer of claim 30, wherein the sensor comprises an absolute barometric pressure sensor.

32. A spirometer, comprising:
a flow chamber defining a pathway, the flow chamber having an inlet and an outlet;
a barrier defining a volumetric area, the barrier positioned within the flow chamber to obstruct a portion of the pathway;
a sensor positioned between the inlet and the barrier; and
a flap movably connected to a portion of the barrier, the flap movable between a closed position and an open position.

33. The spirometer of claim 32, wherein the sensor comprises an absolute barometric pressure sensor.

34. The spirometer of claim 32, wherein, with the flap in the closed position, the flap covers a portion of the pathway adjacent the barrier.

35. The spirometer of claim 32, wherein, with the flap in the open position, the flap is pivoted away from the barrier.

36. The spirometer of claim 32, wherein the flap is formed of a flexible material.

37. The spirometer of claim 32, wherein the barrier and the flap form a unitary component, the flap movably connected to the barrier via a living hinge.
